(19) United States
(12) Patent Application Publication
(10) Pub. No.: US 2021/0136345 A1
(43) Pub. Date: May 6, 2021

(54) SINGLE SENSOR IMAGER

(71) Applicant: TransEnterix Surgical, Inc., Morrisville, NC (US)

(72) Inventor: Bradley Scott Thomas, Cary, NC (US)

(21) Appl. No.: 17/064,144

(22) Filed: Oct. 6, 2020

(51) Int. Cl. H04N 13/229; G02B 5/04; G03B 9/08; G03B 35/02

(52) U.S. Cl. CPC H04N 13/229; G02B 5/04; A61B 90/361; G03B 35/02; G03B 9/08

(57) ABSTRACT

A three dimensional imager which may be used in surgery comprises a single image sensor. First and second lens assemblies are distal to the sensor, and first and second optical wedges are positioned to angle an image their respective lens assembly onto the image sensor. Shutters or polarizers are disposed between each optical wedge and its corresponding lens assembly.

### BACKGROUND

The disclosed invention relates to a three-dimensional imager for use in surgery or other procedures.

### BRIEF DESCRIPTION OF THE DRAWINGS

FIG. 1 is a schematic illustration of a first embodiment of an imaging head for three-dimensional imaging.

FIG. 2 is a schematic illustration of a second embodiment of an imaging head for three-dimensional imaging.

### DETAILED DESCRIPTION

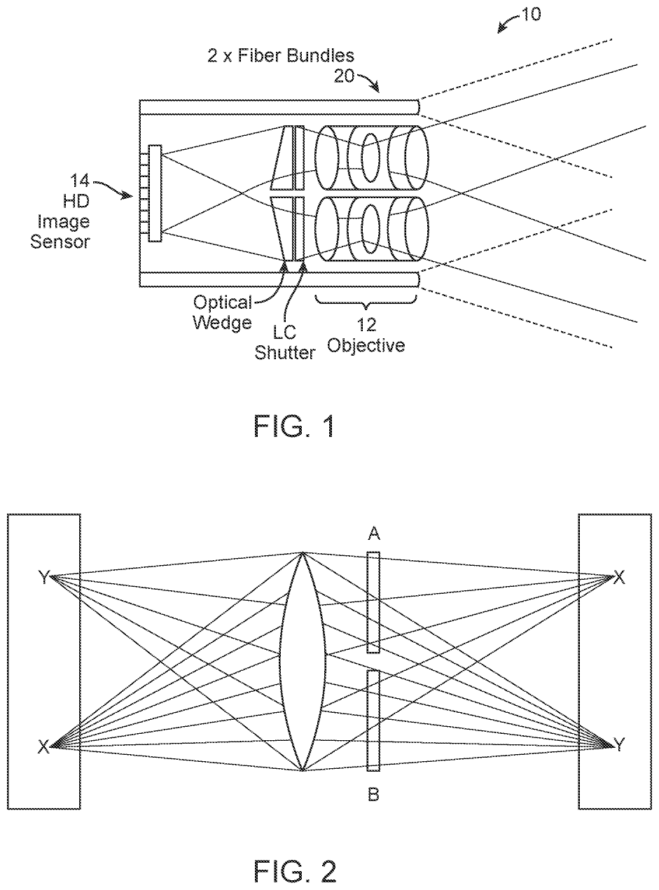

FIG. 1 schematically shows an imaging head 10. A pair of objective lens assemblies 12 are positioned in a distal part of the imaging head 10. A single high definition image sensor 14 is set back from the lens assemblies 12 within the imaging head. At the proximal end of each lens assembly is a prismatic element or optical wedge 16 that angles the image to the center of the image sensor 14. Each optical wedge 16 has a liquid crystal shutter 18 positioned adjacent to, or forming, its distal face. The shutters 18 are alternatingly operated by system electronics at a selected frequency (e.g. 30 Hz or 60 Hz) so that the image captured by the sensor 14 alternates between left eye and right eye images, providing three-dimension stereoscopic vision.

The system includes illumination elements 20 for illuminating the field of view. These may be fiberoptic bundles as shown, or LED chip-on-tip elements, as non-limiting examples.

The illustrated system allows for construction of smaller diameter imaging heads than what is possible using more conventional systems that have a pair of lens assemblies where each directs an image onto a dedicated image sensor. Such systems require relatively large diameter imaging heads due to the need to position the two image sensors side-by-side, which then necessitates a large lens center spacing to allow imaging at short working distances.

In a modified embodiment, the liquid crystal shutter 18 is replaced with polarizers. In this modified embodiment, two LED light sources are orthogonally polarized and alternated to create alternating left eye and right eye images. See FIG. 2.

As yet another alternative, rather than using two lens assemblies and shuttering back and forth between them, an alternative system might make use of an active prism behind a single lens assembly so as to repeatedly redirect the line of sight from the lens system.

What is claimed is:

1. A three dimensional imager comprising:
a single image sensor;
a first lens assembly distal to the image sensor, an optical wedge positioned to angle an image from the first lens assembly onto a center region of the image sensor, and a shutter disposed between the optical wedge and first lens assembly;
a second lens assembly distal to the image sensor and parallel to the first lens assembly, a second optical wedge positioned to angle an image from the second lens assembly onto the center region of the image sensor, and a second shutter disposed between the second optical wedge and second lens assembly.

2. The imager of claim 1, wherein the shutters are liquid crystal shutters.

3. An imaging method comprising the step of providing an imager in accordance with claim 1, and alternating shuttering of the first and second shutters.

4. The method of claim 3, wherein the first and second shutters are alternated at 30 or 60 Hz.

5. A three dimensional imager comprising:
a single image sensor;
a first lens assembly distal to the image sensor, an optical wedge positioned to angle an image from the first lens assembly onto a center region of the image sensor, and a first polarizer disposed between the optical wedge and first lens assembly;
a second lens assembly distal to the image sensor and parallel to the first lens assembly, a second optical wedge positioned to angle an image from the second lens assembly onto the center region of the image sensor, and a second polarizer disposed between the second optical wedge and second lens assembly.

6. A three dimensional imager comprising:
a single image sensor;
lens assembly distal to the image sensor and an active prism between the lens assembly and the image sensor, the prism operable to cyclically redirect the line of sight of the lens assembly.
